(19) United States
(12) Patent Application Publication
(10) Pub. No.: US 2007/0148651 A1
(43) Pub. Date: Jun. 28, 2007

(54) Method and kit for the isolation of rna

(76) Inventors: Uwe Michelsen, Weinheim (DE); Karl Holschuh, Seeheim-Jugenheim (DE); Achim Schwammle, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/582,637

(22) PCT Filed: Nov. 12, 2004
(86) PCT No.: PCT/EP04/12819

§ 371 Date: Jun. 12, 2006

(30) Foreign Application Priority Data
Dec. 12, 2003 (DE) 103 58 137.5

(51) Int. Cl. C12Q 1/68; C12N 1/08

(52) U.S. Cl. 435/6; 435/270; 977/924

(57) ABSTRACT

The present invention relates to a method and a kit for the isolation of RNA in the presence of DNA. The isolation is carried out by binding to a magnetite solid phase, where the binding buffer produces a concentration of guanidinium thiocyanate of>2.5 mol/l, and a certain concentration of phosphate is present during the binding.

The invention relates to a method and a kit for the isolation of RNA in the presence of DNA by specific binding to magnetite supports.

The enrichment or purification and isolation of various nucleic acid species, such as double-stranded plasmid DNA, chromosomal DNA, single-stranded DNA, DNA fragments or RNA, is of central importance in molecular biology. A multiplicity of methods has long been known for separating the various DNA species which may occur in a sample from one another. Use is made here of methods on a purely liquid-chemical basis or various methods with the aid of specifically modified solid phases for binding the nucleic acids.

The analysis of RNA has experienced particular interest in recent years since, in particular, RNA molecules, due to their variety of functions, reflect the biological state of a cell. On the other hand, important pathogenic viruses are provided with RNA genomes, which need to be determined quantitatively and qualitatively by means of molecular diagnostics.

U.S. Pat. No. 4,843,155 describes a purely liquid-chemical method for the specific isolation of RNA molecules. The method utilises the different dissolution behaviour of DNA and RNA. However, highly toxic substances, such as phenol or chloroform, are used here. In addition, time-consuming precipitation reactions with alcohol and centrifugations have to be carried out.

U.S. Pat. No. 5,234,809 and U.S. Pat. No. 6,355,792 describe the use of a solid phase for the isolation of DNA and RNA. It is not possible to distinguish between the two nucleic acid species.

WO 92/18514 and WO 01/46404 describe similar methods for the isolation of DNA/RNA in which metallic oxides, such as, for example, magnetite, are employed as solid phase.

It has now been found that, under certain chaotropic conditions, unmodified magnetite particles bind RNA molecules specifically and effectively, while DNA molecules remain in the supernatant. This is particularly surprising since the literature (M. J. Davies et al., Analytical Biochemistry 262, 92-94 (1998), WO 92/18514 and WO 01/46404) uses precisely the same magnetite supports for the isolation of DNA.

The present invention therefore relates to a method for the isolation of RNA from samples, characterised by the following method steps:
a) provision of a magnetite solid phase;
b) provision of a binding buffer which comprises guanidinium thiocyanate (GTC) in a concentration which, after mixing with the sample, produces a final concentration of >2.5M guanidinium thiocyanate;
c) preparation of a mixture of the sample, the magnetite solid phase and the binding buffer, where a phosphate concentration which supports the binding of RNA is present in this mixture;
d) isolation of the solid phase with the bound RNA.

In a preferred embodiment, after isolation of the solid phase with the bound RNA (step d)),
e) the said solid phase is optionally washed and
f) the RNA is eluted from the solid phase.

In a preferred embodiment, the elution in step e) is carried out using elution buffers which facilitate a pH range >7 and comprise phosphate.

In a further preferred embodiment, the binding buffer additionally comprises chelators, such as EDTA.

In a preferred embodiment, the solid phase consists of particulate magnetite. Particular preference is given to magnetite particles having a diameter of 0.01 to 2 μm and a specific surface area of 1 to 100 m²/g.

The present invention also relates to a kit for the isolation of RNA by the method according to the invention, at least comprising a magnetite solid phase and a binding buffer having a GTC concentration of greater than 3 mol/l.

In a preferred embodiment, the binding buffer comprises at least between 4 and 8 mol/l of GTC, between 5 and 200 mmol/l of EDTA and typically between 10 and 100 mmol/l of Tris HCl or similar buffer substances.

In a preferred embodiment, the kit additionally comprises one or more of the following constituents:
an elution buffer
a wash buffer
a phosphate salt solution.

For the purposes of the invention, samples are samples of any type in which RNA is suspected. The sample can be of synthetic or preferably genetic-engineering, biotechnological or biological origin, i.e., for example, from bacteria, viruses, body cells, blood, plasma, cerebrospinal fluid, urine, faeces, milk, tissue, fermentation broth or cell cultures.

The sample can be employed directly or, if necessary, firstly digested. For example, samples which comprise cells or viral particles firstly have to be digested in order to liberate the RNA from the cells or particles. Suitable methods for the digestion of the samples or lysis of cells are known to the person skilled in the art.

RNA is any natural or synthetic form of ribonucleic acid, in particular m-RNA, t-RNA, ribosomal RNA, viral RNA, or synthetic RNA molecules, such as siRNA (RNAi).

In accordance with the invention, the term “phosphate” encompasses inorganic phosphate and organic phosphate. Examples thereof are phosphate salts, such as sodium hydrogenphosphate (inorganic phosphate), or phosphate-containing hydrocarbon compounds, such as amino acids, creatine phosphates and phosphate-containing proteins (organic phosphate).

A magnetite solid phase is a support whose surface consists at least for the most part, preferably completely, of magnetite (Fe₃O₄). The solid phase can be, for example, in the form of a plate, particle, coating, fibre, filter or another, preferably porous structure. The solid phase particularly preferably consists of magnetite particles.

Various production processes are known for the production of magnetite particles. Examples are disclosed in:
Massart, IEEE Trans. Magn. 17, 1247-1248 (1981)
Sugimoto, Matijevic, J. Colloid Interface Sci. 74, 227-243 (1980)
Qu et al., J. Colloid Interface Sci. 215, 190-192 (1999)

The magnetite solid phase is particularly preferably produced by the method of Sugimoto and Matijevic.

In general, 1 mg of such particles binds about 10 μg of RNA. Since most samples comprise less than 1 μg of RNA, 0.5 to 5 mg, preferably about 1 mg, of particles are typically employed for the isolation of RNA in accordance with the invention.

The essence of the present invention is that RNA binds to magnetite solid phases in aqueous solutions in the presence of guanidinium thiocyanate (GTC) and phosphate, while DNA remains in solution. In this way, more than 70%, frequently more than 90%, of the RNA present in the sample can generally be isolated specifically.

In order to ensure the binding of RNA, the aqueous binding buffer must facilitate a concentration of at least 2.5M GTC, preferably 3-5M GTC, in the mixture with the sample and the solid phase. To this end, use is preferably made of binding buffers having a concentration of greater than 3M, preferably 5 to 8M GTC, depending on the volume ratio of the sample and of the binding buffer.

The specific binding of RNA takes place under suitable conditions in a broad pH range. The binding buffer preferably establishes a pH range of 6-10, particularly preferably of pH 7-9. The binding buffer therefore comprises buffer substances which facilitate this. Suitable buffer substances are known to the person skilled in the art. Examples thereof are Tris HCl, Tricine, MOPS and others.

A prerequisite for the specific isolation of RNA is the presence of a certain amount of phosphate during the binding to the magnetite solid phase. If no phosphate is present during the binding (i.e. during mixing of the sample with the binding buffer and the solid phase), both DNA and RNA generally bind to the magnetite. If too much phosphate is present during the binding, neither DNA nor RNA bind.

The amount of phosphate necessary to support the specific binding of RNA depends on the size of the surface of the magnetite solid phase. The reason for this correlation is probably an interaction between the surface of the solid phase and the phosphate. The amount of phosphate must be sufficiently large in order to prevent the binding of DNA to the surface of the solid phase, but must not be so large that the phosphate interferes with the binding of RNA.

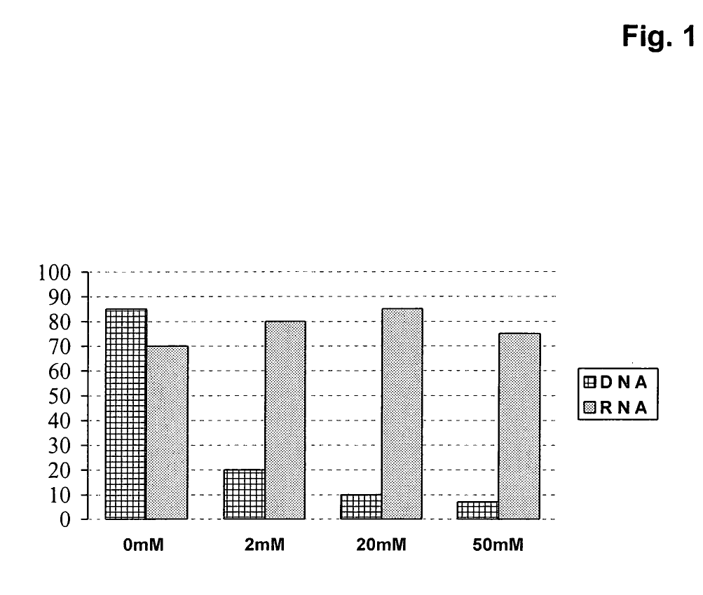

As shown by way of example in FIG. 1, a concentration of 20 mM phosphate per mg of the magnetite particles preferably employed (Merck Mag-Prep® magnetite, Art. No. 101882, i.e. particles having a diameter of about 1 μm, a specific surface area of about 20 m²/g, produced by the method of Sugimoto, Matijevic, J. Colloid Interface Sci. 74, 227-243 (1980)) effects specific and efficient binding of RNA to the magnetite solid phase. The DNA remains in solution.

For efficient binding of the RNA, the phosphate concentration should not be greater than 200 mM per mg of the magnetite particles used.

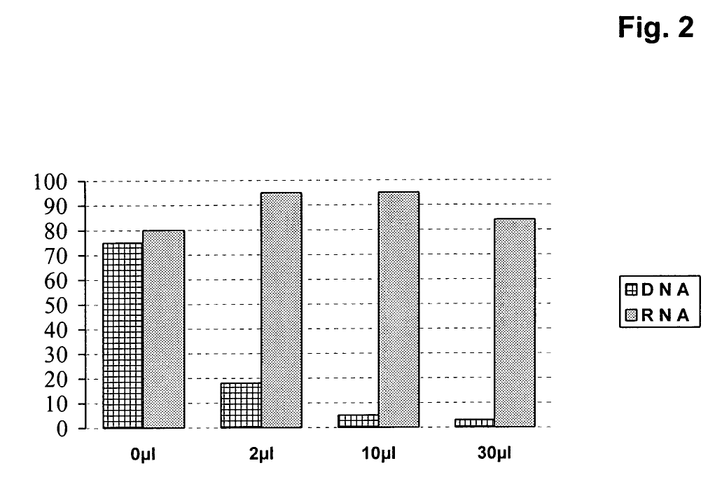

The amount of phosphate necessary to support the specific binding of RNA can be added to the mixture through corresponding additions to the binding buffer. Equally, however, the sample may itself also already comprise phosphate, as is known for body fluids, such as urine or blood plasma, which comprise phosphate in inorganic and in protein-bound form. In this case, the requisite amount of phosphate for specific RNA binding can be set through the mixing ratio of sample and binding buffer and/or the amount of magnetite. This is shown by way of example in FIG. 2.

For the purposes of the invention, the statement that the mixture of binding buffer, aqueous sample and solid phase comprises a phosphate concentration which “supports the specific binding of RNA” thus means that the mixture comprises sufficient inorganic and/or organic phosphate so that binding of DNA is prevented and binding of RNA is supported.

In the case of samples of biological origin, the nucleic acid-containing cells or viruses have to be lysed in order quantitatively to enrich the RNA in accordance with the method according to the invention. In order to support the lysis, the person skilled in the art uses, inter alia, non-ionic detergents, such as NP 40, Tween® 20 or Triton® X-100. These non-ionic detergents may adversely affect the efficiency of the RNA isolation according to the invention. However, it has been found that this adverse effect is overcome by addition of chelators, such as EDTA.

In accordance with the invention, the binding buffer and/or the sample therefore preferably additionally comprise chelators, such as EDTA. The concentration of chelators during the binding is preferably between 5 and 200 mmol/l. The binding buffer therefore typically comprises between 5 and 200 mmol/l, preferably between 10 and 100 mmol/l, of chelators. In addition, the binding buffer may comprise further substances, such as stabilisers, enzyme inhibitors, etc.

In order to carry out the method according to the invention, the sample is typically firstly mixed with the binding buffer, and the solid phase is then added. The suspension of sample, binding buffer and solid phase is mixed well.

After an incubation time of 3 to 60 minutes, typically about 10 minutes, the supernatant is carefully separated off.

The solid phase is optionally washed, for example with an acidic wash buffer, as disclosed, for example, in U.S. Pat. No. 6,355,792.

If isolation of the RNA is desired, the solid phase, after re-separation and removal of the supernatant, is resuspended in the elution buffer, mixed well and incubated for 3 to 60, typically 10, minutes. After the solid phase has been separated off, the supernatant comprising the RNA can be used for further analysis.

The present invention also relates to a kit for the isolation of RNA by means of the method according to the invention. The kit comprises a magnetite solid phase and a binding buffer having a concentration of at least 3 mol/l of GTC. Furthermore, the kit preferably additionally comprises wash buffer and elution buffer and a phosphate salt solution for setting the requisite phosphate content of a sample.

In a preferred embodiment, the binding buffer comprises at least GTC in a concentration between 4 and 8 mol/l, EDTA in a concentration between 5 and 200 mmol/l and, as buffer substance, typically Tris HCl in a concentration between 10 and 100 mmol/l.

In a preferred embodiment, the kit comprises, as solid phase, magnetite particles produced by the method of Sugimoto, Matijevic, J. Colloid Interface Sci. 74, 227-243 (1980).

The method according to the invention and the kit according to the invention thus provide for the first time an effective and quantitative possibility for the solid phase-supported isolation of RNA from samples in the presence of DNA.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application DE 10358137.5, filed on Dec. 12, 2003, is incorporated into this application by way of reference.

### EXAMPLES

### Example 1

1 mg of MagPrep® magnetite particles (Merck KGaA) are mixed with a mix of 500 μl of binding buffer B (5M guanidinium thiocyanate+50 mM Tris HCl pH 7.5+20 mM EDTA+1% of Triton®X 100), DNA (200 ng of linearised plasmid DNA) and RNA (200 ng of 16S/23S RNA). Increasing amounts of sodium phosphate were addedto -the mix in advance.

After incubation for 10 minutes, the mixture is magnetised, and the supernatant is carefully separated off. After removal of the magnetic field, the particles are resuspended with 500 μl of wash buffer AT (10 mM acetate/Tris pH 4.0) and incubated at room temperature for up to 10 minutes. After magnetisation, the supernatant is carefully removed, and 500 μl of wash buffer AT are again added. After removal of the magnetic field, the particles are resuspended and magnetised, and the supernatant is discarded. The elution of the nucleic acids from the particles is carried out after incubation for 10 minutes in 100 μl of elution buffer P (10 mM Tris HCl pH 8.5+1 mM EDTA+50 mM sodium hydrogenphosphate) at 50° C. After magnetisation, the eluate is transferred into a fresh, sterile vessel. The concentration of DNA and RNA is quantified by fluorescence measurement using PicoGreen® and RiboGreen® (Molecular Probes) in accordance with the manufacturer's instructions. FIG. 1 shows the yield of DNA and RNA as a function of the phosphate concentration. The phosphate concentration is shown on the abscissa, and the recovery rate of DNA and RNA in % is shown on the ordinate.

It can clearly be seen that the binding of DNA drops considerably even in the presence of small amounts of phosphate. By contrast, the RNA binds efficiently in the presence of 2 to 50 mM phosphate.

### Example 2

1 mg of MagPrep® magnetite particles (Merck KGaA) are mixed with a mix of 500 μl, of binding buffer B (5M guanidinium thiocyanate+50 mM Tris HCl pH 7.5+20 mM EDTA+1% of Triton®X 100), DNA (200 ng of linearised plasmid DNA) and RNA (200 ng of 16S/23S RNA). Increasing amounts of human plasma were added to the mix in advance.

The further steps are described in Example 1.

FIG. 2 shows the yield of DNA and RNA as a function of the plasma concentration. The amount of added plasma is shown on the abscissa, and the recovery rate in % is shown on the ordinate.

Since plasma comprises phosphorylated proteins and inorganic phosphate, the same picture arises on addition of plasma as on addition of inorganic phosphate (Example 1). This shows that in many cases in which the sample already comprises inorganic and/or organic phosphate, further addition of phosphate salts is not necessary.

### Example 3

1 mg of MagPrep® magnetite particles (Merck KGaA) are mixed with 500 μl of four different binding buffers:

5M guanidinium thiocyanate (GTC) or

5M guanidinium HCl (GHCl) ) or

5M sodium thiocyanate (NaTC) or

5M sodium perchlorate, (NaClO).

All binding buffers comprise DNA (200 ng of linearised plasmid DNA) and RNA (200 ng of 16S/23S RNA) and 3 mg/ml of bovine serum albumin.

The further steps are described in Example 1.

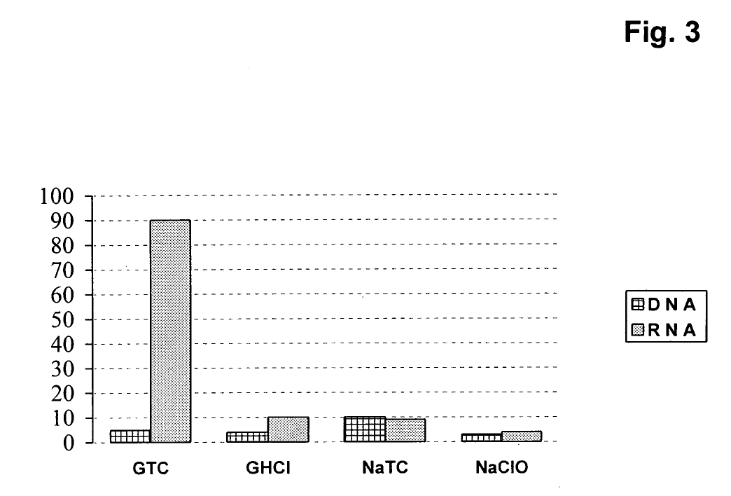

FIG. 3 shows the yield of DNA and RNA as a function of the chaotropic salt employed. Specific binding of RNA only takes place in the presence of guanidinium thiocyanate.

1. Method for the isolation of RNA from samples, characterised by the following method steps:
a) provision of a magnetite solid phase;
b) provision of a binding buffer which comprises guanidinium thiocyanate in a concentration which, after mixing with the sample, produces a final concentration of >2.5M guanidinium thiocyanate;
c) mixing of the sample with the magnetite solid phase and the binding buffer, where a phosphate concentration which supports the binding of RNA is present in this mixture;
d) isolation of the solid phase with the bound RNA.

2. Method according to claim 1, characterised in that, after step d), the solid phase is optionally washed, and the RNA is subsequently eluted from the solid phase.

3. Method according to claim 2, characterised in that the elution is carried out using elution buffers which facilitate a pH range >7 and comprise phosphate.

4. Method according to claim 1, characterised in that the binding buffer additionally comprises chelators, such as EDTA.

5. Method according to claim 1, characterised in that the solid phase consists of magnetite particles having a diameter of 0.01 to 2 μm and a specific surface area of 1-100 m²/g.

6. Kit for the isolation of RNA by the method according to claim 1, at least comprising a magnetite solid phase and a binding buffer having a GTC concentration of greater than 3 mol/l.

7. Kit according to claim 6, characterised in that the binding buffer comprises at least between 4 and 8 mol/l of GTC and between 5 and 200 mmol/l of EDTA.

8. Kit according to claim 6, characterised in that the kit additionally comprises one or more of the following constituents:
an elution buffer
a wash buffer
a phosphate salt solution.
